(19) United States
(12) Patent Application Publication
(10) Pub. No.: US 2021/0093755 A1
(43) Pub. Date: Apr. 1, 2021

(54) DEVICE FOR ORAL-TO-NASAL REPOSITIONING OF NASOBILIARY DRAINAGE TUBE

(71) Applicant: KOREA UNIVERSITY RESEARCH AND BUSINESS FOUNDATION, Seoul (KR)

(72) Inventors: Hong Sik Lee, Seoul (KR); Jae Min Lee, Seoul (KR)

(21) Appl. No.: 16/606,393

(22) PCT Filed: May 23, 2018
(86) PCT No.: PCT/KR2018/005823

§ 371 Date: Oct. 18, 2019

(30) Foreign Application Priority Data
May 24, 2017 (KR) 10-2017-0064098
May 17, 2018 (KR) 10-2018-0056442

(51) Int. Cl. A61M 1/00

(52) U.S. Cl. CPC A61M 1/008; A61M 2210/065

(57) ABSTRACT

The present disclosure relates to a device for oral-to-nasal repositioning of a nasobiliary drainage tube, including a ring type module which is inserted into an oral cavity to reposition the nasobiliary drainage tube from mouth to nose, and a nasal tube which is inserted into pharynx through a nasal cavity, taken out of the mouth by the ring type module and connected with the nasobiliary drainage tube, wherein the ring type module includes a snare shaped ring part which is inserted into the pharynx through the oral cavity, and a ring handle connected to an end of the ring part.

### TECHNICAL FIELD

The present disclosure relates to an instrument that aids in nasobiliary drainage, and more particularly, to a device for oral-to-nasal repositioning of a nasobiliary drainage tube to reposition the nasobiliary drainage tube from mouth to nose safely, easily and quickly in nasobiliary drainage using an endoscopy.

### BACKGROUND ART

In the existing endoscopic retrograde cholangiopancreatography, physicians perform a repositioning procedure of a nasobiliary drainage tube from mouth to nose using forceps or fingers.

The endoscopic nasobiliary drainage (ENBD) is a technique widely used in many diseases, for example, choledocholithiasis, cholangiocarcinoma, cholangitis, duodenal papilla or biliary atresia, and oral-to-nasal repositioning of a nasobiliary drainage tube is an essential process. Immediately after an endoscopic surgery, the drainage tube goes out through the mouth, and repositioning the drainage tube from the mouth to the nose is an essential but tedious process. The existing repositioning method includes pulling a plastic tube out of the mouth through the nasal cavity, connecting a drainage tube and allowing the plastic tube to pass back through the nasal cavity so as to move the drainage tube to the nose.

That is, in general, to reposition a nasobiliary drainage tube, first, a soft nasal tube is inserted into the nose, the end of the nasal tube positioned in the oral cavity is held using fingers or forceps, the nasal tube is pulled out of the mouth, the nasobiliary drainage tube is connected to the nasal tube, and the nasal tube is pulled back from the nose to draw the nasobiliary drainage tube through the nostril.

In detail, the existing process of repositioning a nasobiliary drainage tube is described with reference to FIG. 5.

As shown in the picture 1) of FIG. 5, first, an ENBD tube goes out through the mouth, as shown in the picture 2) of FIG. 5, a short plastic tube is inserted into the nose such that the end is placed behind the uvula, as shown in the picture 3) of FIG. 5, a mouthpiece is removed from the open mouth, and the end of the plastic tube is pulled out of the mouth using fingers or forceps, and as shown in the picture 4) of FIG. 5, the ENBD tube is inserted into the short plastic tube. Subsequently, as shown in the picture 5) of FIG. 5, the plastic tube is pulled out of the nose, and as shown in the picture 6) of FIG. 5, the ENBD tube moved to the nose is tightly pulled up, and then the short plastic tube is removed.

In the step of placing the short plastic tube behind the uvula and pulling the end of the plastic tube using fingers, there is no available surgical assistant instrument now, so the physician has to put fingers deep in the mouth, hold the plastic tube in reliance on the sense of the fingertips or forcibly open the mouth using forceps and a medical penlight, then hold and pull the plastic tube.

In this case, inconveniently, the physician needs to check the location of the end of the nasal tube by seeing with eyes or touching with fingers, and because it is necessary to remove the mouthpiece for surgery, the physician may be injured by the patient's teeth, the patient may unconsciously injure the physician's hand by the teeth during surgery, and the patient's oral cavity and teeth may be injured by the forceps.

Additionally, because the surgery is possible in the patient's open mouth, the physician has to wait for the patient to wake up after removing the mouthpiece from the mouth for the endoscopy, and the surgery is impossible in the presence of the mouthpiece.

As described above, damage of the patient's teeth and oral mucosa, procedure time delays, procedure failures, dislocation of the nasobiliary drainage tube and the physician's finger injuries occur frequently during surgery, but there is no solution now.

### DISCLOSURE OF THE INVENTION

### Technical Problem

The present disclosure is designed to solve the above-described problem, and therefore the present disclosure is directed to providing a device for oral-to-nasal repositioning of a nasobiliary drainage tube to reposition the nasobiliary drainage tube from mouth to nose safely, easily and quickly, in nasobiliary drainage using an endoscopy.

Meanwhile, other objects not specified herein will be additionally considered in the range that can be easily inferred from the following detailed description and its effects.

### Technical Solution

To achieve the above-described object, a ring type module according to the present disclosure includes a ring part which is inserted into pharynx through an oral cavity, and a ring handle connected to an end of the ring part, wherein when an end of a nasal tube inserted into the pharynx through a nasal cavity passes through the ring part positioned in the pharynx, the nasal tube is taken out of mouth by pulling the ring handle.

To achieve the above-described object, a device for oral-to-nasal repositioning of a nasobiliary drainage tube according to the present disclosure includes a ring type module which is inserted into an oral cavity to reposition the nasobiliary drainage tube from mouth to nose, and a nasal tube which is inserted into pharynx through a nasal cavity, taken out of the mouth by the ring type module and connected with the nasobiliary drainage tube, wherein the ring type module includes a snare shaped ring part which is inserted into the pharynx through the oral cavity, and a ring handle connected to an end of the ring part.

The ring handle is provided in a shape of a flat bar, and the ring part curves down from an end of the ring handle.

The device for oral-to-nasal repositioning of a nasobiliary drainage tube further includes an adjuster slidably moveably provided in the ring handle connected to the end of the ring part, to adjust a size of the ring part protruding from the end of the ring handle with sliding movement.

The nasal tube is formed of a hollow elongated flexible tube, and has a multistep third mark at a point.

To achieve the above-described object, a method of using the device for oral-to-nasal repositioning of a nasobiliary drainage tube according to the present disclosure includes pushing and inserting the ring part of the ring type module into a user's mouth through a hole of a mouthpiece placed in the mouth, pushing and inserting the nasal tube into nose to allow the flexible tube to pass through the ring part, pulling the ring type module out of the mouth to take out the nasal tube caught in the ring part of the ring type module, and connecting the nasobiliary drainage tube to the nasal tube pulled out as caught in the ring part and pulling the nasal tube out of the nose, wherein in the insertion of the ring part into the mouth, a size of the ring part is adjusted using a length adjuster provided in the ring handle.

### Advantageous Effects

The device for oral-to-nasal repositioning of a nasobiliary drainage tube according to the present disclosure can reposition the nasobiliary drainage tube from mouth to nose safely, simply and efficiently using the ring type module which is inserted into the oral cavity such that the end is positioned in the pharynx to reposition the nasobiliary drainage tube from the mouth to the nose, and the nasal tube which is inserted into the nasal cavity through the nostril, pulled out of the mouth by the ring type module and connected with the nasobiliary drainage tube.

The present disclosure can adjust the size of the ring part through the adjuster installed in the ring handle to fit the patient's physical characteristics such as gender, body size or height.

The present disclosure inserts the ring part into the oral cavity while the flat bar type ring handle presses the tongue when inserting the ring part into the oral cavity and the pharynx, creating a sufficient space of the oral cavity, so the ring part can be smoothly inserted into the oral cavity and the pharynx.

The present disclosure has the elastic ring part bent downward to form a smooth curve, allowing the ring part to smoothly pass from the oral cavity to the pharynx, so the ring part can be stably inserted into the pharynx and positioned in place.

The present disclosure can reduce the procedure time, minimize the risks of teeth/oral mucosa damage, and prevent the physician's injuries.

The present disclosure avoids the risks of infection caused by the use of fingers and forceps.

The present disclosure can perform the procedure while the patient is asleep with the mouthpiece placed in the mouth.

The present disclosure provides economic efficiency by virtue of disinfection and reusability.

### BRIEF DESCRIPTION OF THE DRAWINGS

FIG. 1 is a perspective view of a ring type module of a device for oral-to-nasal repositioning of a nasobiliary drainage tube according to an embodiment of the present disclosure.

FIG. 2 shows schematic plane views showing the size adjustment of a ring part by operation of the ring type module of FIG. 1.

FIG. 3 is a perspective view of a nasal tube of a device for oral-to-nasal repositioning of a nasobiliary drainage tube according to an embodiment of the present disclosure.

FIG. 4 shows schematic diagrams showing a procedure using a device for oral-to-nasal repositioning of a nasobiliary drainage tube according to the present disclosure.

FIG. 5 shows schematic diagrams showing an oral-to-nasal repositioning procedure of a nasobiliary drainage tube according to the related art.

### MODE FOR CARRYING OUT THE INVENTION

Hereinafter, embodiments of the present disclosure will be described in further detail with reference to the accompanying drawings. However, the present disclosure is not limited to the disclosed embodiments and will be embodied in a variety of different forms, and these embodiments make the present disclosure complete and are provided to help those skilled in the art to fully understand the scope of the present disclosure. In the drawings, identical reference signs indicate identical elements.

In adding the reference signs to the elements in each drawing, it should be noted that the identical elements are given the identical reference signs as possible, they are shown on different drawings though. Additionally, in describing the present disclosure, when it is deemed that a certain detailed description of relevant known elements or functions may make the key subject matter of the present disclosure ambiguous, its detailed description is omitted herein.

Additionally, the terms first, second, A, B, (a) and (b) may be used to describe the elements of the present disclosure. These terms are used to distinguish one element from another, and the essence, order or sequence of the stated elements is not limited by the terms. It should be understood that when an element is referred to as being “connected’, “coupled” or “linked” to another element, the element may be directly connected, coupled or linked to another element, but other elements may be “connected”, “coupled” or “linked” between each element.

Hereinafter, configuration and operation of a device for oral-to-nasal repositioning of a nasobiliary drainage tube according to an embodiment of the present disclosure will be described in detail with reference to the drawings.

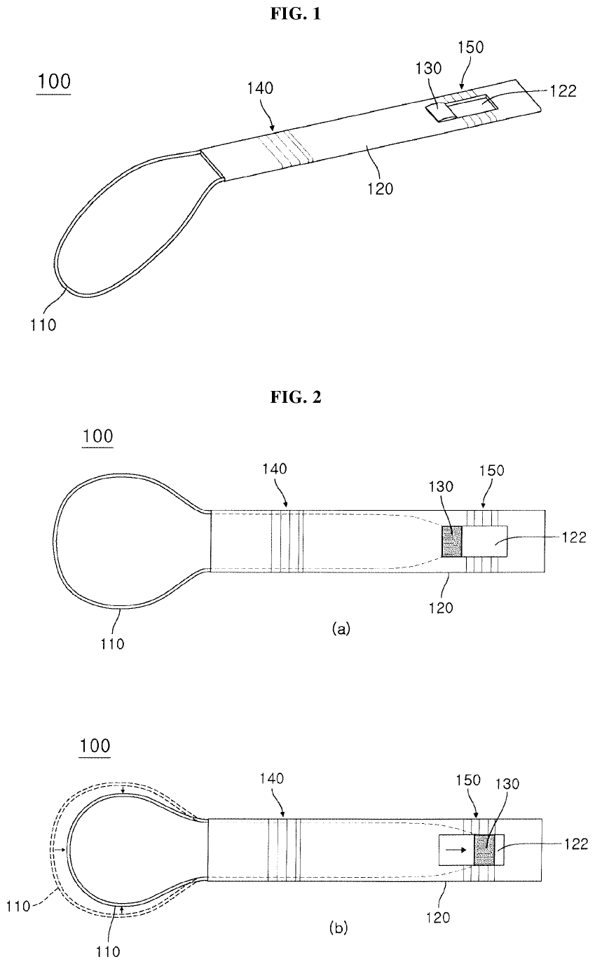

Referring to FIGS. 1 to 4, the device for oral-to-nasal repositioning of a nasobiliary drainage tube according to an embodiment of the present disclosure includes a ring type module 100 which is inserted into the oral cavity such that the end is positioned in the pharynx to reposition the nasobiliary drainage tube 10 from the mouth to the nose, and a nasal tube 200 which is inserted into the nasal cavity through the nostril, pulled out of the mouth by the ring type module 100 and connected with the nasobiliary drainage tube 10.

Referring to FIGS. 1, 2 and 4, the ring type module 100 is used to take the nasal tube 200 connected to the nasobiliary drainage tube 10 out of the mouth from the nose, to reposition the nasobiliary drainage tube 10 from the mouth to the nose. The ring type module 100 includes a ring part 110 having an end that is inserted into the pharynx through the oral cavity, a ring handle 120 connected to the rear end of the ring part 110, and an adjuster 130 provided in the ring handle 120 to adjust the size of the ring part 110.

The ring part 110 is made of a wire material having elastic property. The ring part 110 is a ring type, and is formed in the shape of a snare having a hollow space inside. The ring part 110 may be formed with a predetermined diameter, and it is most desirable that the ring part 110 is formed approximately 8-12 cm in diameter. The ring part 110 is inserted into the oral cavity and is disposed in the pharynx in part or in whole. The ring part 110 is connected while being bent downward at the angle of approximately 15-30° from the front end of the ring handle 120. Advantageously, the ring part 110 bent downward is more stably inserted and positioned into the patient's oral cavity and pharynx of curved shape. Meanwhile, each patient differs in the size and depth of the oral cavity and the pharynx according to the physical characteristics such as gender, body size or height. Accordingly, the ring part 110 according to an embodiment of the present disclosure has an adjustable size from the front end of the ring handle 120.

The size of the ring part 110 may be adjusted by the adjuster 130 provided in the ring handle 120. The size of the ring part 110 is adjusted by adjusting an area exposed from the front end of the ring handle 120 by the adjuster 130. For example, when the ring part 110 is drawn to a large extent from the front end of the ring handle 120, the size of the ring part 110 increases, and when the ring part 110 is drawn to a small extent from the front end of the ring handle 120, the size of the ring part 110 reduces.

The ring handle 120 is formed in the shape of a flat bar. The ring handle 120 has a predetermined length, and is preferably formed 15 cm in length. The ring part 110 in the shape of a snare is connected to the front end of the ring handle 120. As the rear end of the ring part 110 connected to the ring handle 120 moves into the ring handle 120 in the forward/backward direction, the size of the area exposed from the ring handle 120 is adjusted. The ring handle 120 connected with the ring part 110 is inserted into the oral cavity together with the ring part 110. Because the ring handle 120 has the shape of a flat bar as described above, the ring handle 120 is inserted into the oral cavity while stably pressing the tongue. In the insertion of the ring part 110 into the oral cavity, the tongue is pressed down using the ring handle 120, thereby creating more space of the oral cavity and achieving stable insertion of the ring part 110 into the oral cavity and the pharynx.

Meanwhile, a multistep first marker 140 is provided on the ring handle 120 to adjust the insertion depth of the ring part 110 inserted and positioned into the oral cavity and the pharynx. The first marker 140 is provided in the shape of a plurality of reference lines, taken into account the patient's physical traits such as gender, body size or height. The physician checks the patient's physical traits such as gender, body size or height, and adjusts the insertion depth of the ring handle 120 into the oral cavity using the first marker 140, to stably position the ring part 110 in the patient's pharynx.

In addition, the ring handle 120 has a sliding groove 122 at the rear end, and the adjuster 130 is slidably moveably installed in the sliding groove 122.

The adjuster 130 is slidably moveably provided in the sliding groove 122 of the ring handle 120 to adjust the size of the ring part 110 protruding from the front end of the ring handle 120. In detail, the adjuster 130 is slidably moveably provided in the ring handle 120, and the rear end of the ring part 110 is connected. As part of the ring part 110 having the rear end connected to the adjuster 130 goes in or out of the ring handle 120 with the sliding movement of the adjuster 130, the size of the part protruding from the front end of the ring handle 120 is adjusted.

Meanwhile, a multistep second marker 150 for adjusting the size of the ring part 110 is provided on the ring handle 120 where the adjuster 130 slidably moves. The second marker 150 is provided in the shape of a plurality of reference lines taken into account the patient's physical traits such as gender, body size or height. The physician checks the patient's physical traits such as gender, body size or height, and adjusts the size of the ring part 110 using the second marker 150, to stably insert and position the ring part 110 into the patient's oral cavity and pharynx. The adjuster 130 is installed such that it is repeatedly fixed to and released from the sliding groove 122 of the ring handle 120 by a separate fixing means (not shown).

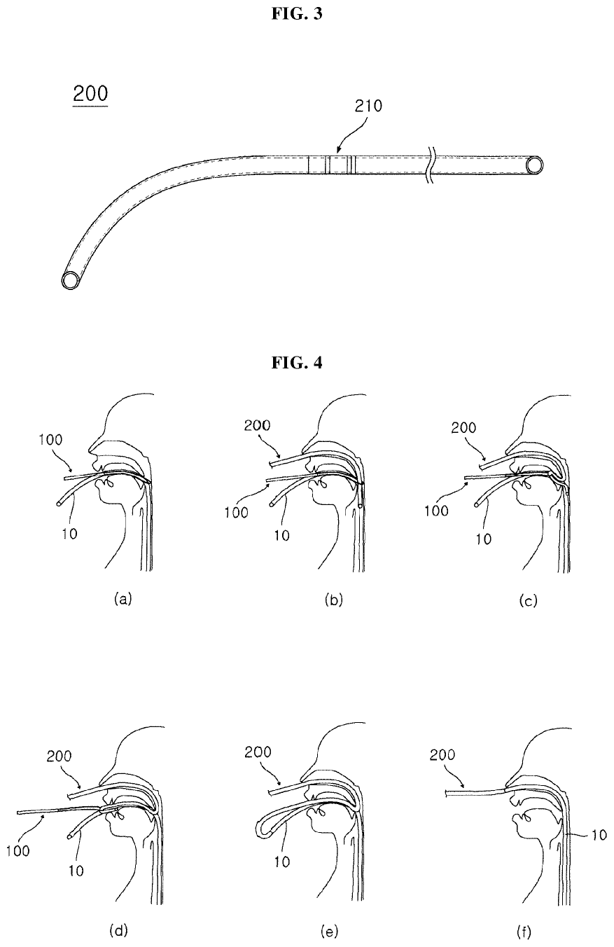

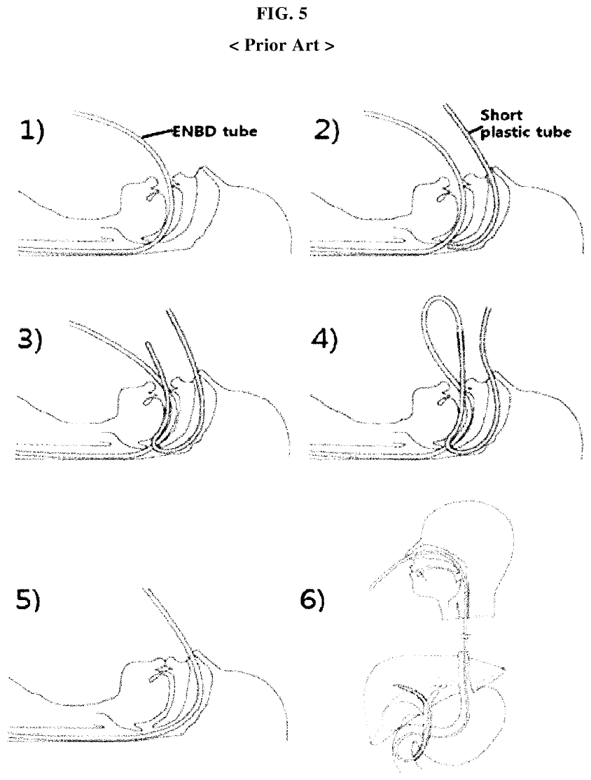

Referring to FIG. 3, the nasal tube 200 is formed in the shape of a hollow elongated flexible tube, and is made of a soft material such as PVC or silicone. A multistep third marker 210 is formed at a point of the nasal tube 200. The third marker 210 is used to measure the insertion depth of the nasal tube 200 according to the patient's physical characteristics such as gender, body size or height, and may be provided in the shape of reference lines by the unit of 15 cm, 20 cm and 25 cm from the rear end.

Meanwhile, in the existing drainage procedure, there is a need to hold a tube having no marker through forceps while checking with eyes or fingers, whereas the nasal tube 200 having the third marker 210 according to the present disclosure can be inserted into the patient's nasal cavity to a proper depth and be positioned in the pharynx without seeing with eyes in the surgery.

With the above-described structure, the ring type module 100 of the device for oral-to-nasal repositioning of a nasobiliary drainage tube according to an embodiment of the present disclosure is configured to stably insert and position the ring part 110 into the patient's oral cavity and pharynx by adjusting the extent of insertion of the ring handle 120 into the oral cavity and the size of the ring part 110 using the first marker 140 and the second marker 150 according to the patient's physical traits such as gender, body size or height. Additionally, more space of the oral cavity is created by pressing the tongue through the flat bar type ring handle 120, which makes the insertion of the ring part 110 easier, and as the ring part 110 curves down to fit the curved shape of the oral cavity, advantageously, the ring part 110 can be inserted and positioned into the patient's oral cavity and pharynx more stably.

Additionally, in the existing procedure, to reroute the nasobiliary drainage tube 10 from mouth to nose, while the patient is asleep, the mouthpiece is removed, the patient's mouth is opened using fingers, and the nasal tube is inserted into the nose and pulled out of the mouth using forceps or fingers, and thus the procedure itself is difficult and is difficult for low skilled physicians to perform. In contrast, the nasobiliary drainage procedure using the device according to an embodiment of the present disclosure can achieve the oral insertion in the presence of the mouthpiece, and thus is possible irrespective of whether the patient is awake or asleep, and allows even low skilled physicians to perform quickly and accurately.

Additionally, in the existing procedure, the oral cavity is frequently damaged by fingers or forceps, whereas the ring type module 100 according to an embodiment of the present disclosure includes the ring part 110 formed of an elastic wire, and thus can minimize the oral mucosa damage compared to the sharp forceps or fingers.

In 2015-2016, the device for oral-to-nasal repositioning according to an embodiment of the present disclosure successfully rerouted nasobiliary drainage tubes from mouth to nose and positioned them in place with a success rate of 99.3% in 300 clinical cases.

The procedure time was within 1 minute, and it is shorter by about 4 minutes than that of the method using fingers, and by about 3 minutes than that of the method using forceps.

Hereinafter, the procedure using the device for oral-to-nasal repositioning of a nasobiliary drainage tube according to an embodiment of the present disclosure will be described with reference to FIG. 4.

First, referring to (a) of FIG. 4, one side of the nasobiliary drainage tube (ENBD tube) 10 is exposed through the patient's opening required for the drainage procedure, and the ring part 110 of the ring type module 100 is pushed and inserted into the oral cavity through the hole of the mouthpiece placed in the patient's opening. In this instance, the insertion depth of the ring part 110 into the oral cavity is adjusted using the first marker 140 of the ring handle 120, taking into account the patient's physical characteristics such as gender, body size or height.

That is, in the step of inserting the ring part 110 into the oral cavity, the insertion depth of the ring part 110 is suitably controlled by adjusting the insertion depth of the ring handle 120 to fit the patient's physical characteristics using the first marker 140 provided in the ring handle 120. Meanwhile, with the insertion of the ring part 110 into the oral cavity, the size of the ring part 110 protruding from the front end of the ring handle 120 is adjusted using the adjuster 130 provided in the ring handle 120. The size of the ring part 110 may be adjusted using the second marker 150 according to the patient's physical characteristics such as gender, body size or height. As described above, the ring part 110 is, in part or in whole, stably positioned in the patient's the pharynx through the oral cavity by the size adjustment by the adjuster 130, and the insertion depth adjustment by the ring handle 120.

After the ring part 110 of the ring type module 100 is stably positioned in the patient's pharynx, as shown in (b) of FIG. 4, the nasal tube 200 is pushed into the nasal cavity through the nostril. The insertion depth of the nasal tube 200 is adjusted using the third marker 210, taking into account the patient's physical characteristics such as gender, body size or height. The nasal tube 200 inserted into the nasal cavity moves to the pharynx along the nasal cavity and passes through the ring part 110 of the ring type module 100.

Referring to (c) of FIG. 4, when the nasal tube 200 passes through the ring part 110, the ring type module 100 is pulled out of the oral cavity to take out the nasal tube 200 caught in the ring part 110 of the ring type module 100. In this case, due to the flexible property of the nasal tube 200, the nasal tube 200 can be tightly caught in the ring part 110.

Referring to (d) of FIG. 4, the nasobiliary drainage tube 10 is connected to the nasal tube 200 pulled out as caught in the ring part 110, and the nasal tube 200 is pulled out of the nose.

Referring to (e) and (f) of FIG. 4, when the nasobiliary drainage tube 10 connected to the nasal tube 200 is taken out of the nose through the process of pulling the nasal tube 200, the nasal tube 200 connected to the nasobiliary drainage tube 10 is separated.

As described above, the device for oral-to-nasal repositioning of a nasobiliary drainage tube according to the present disclosure can reposition the drainage tube from mouth to nose very simply and efficiently in the endoscopic nasobiliary drainage.

Unless the context clearly indicates otherwise, it should be interpreted that the term “comprises”, “includes” or “have” when used in this specification, specifies the presence of stated elements, but does not preclude the presence or addition of one or more other elements. Unless otherwise defined, all terms including technical and scientific terms as used herein have the same meaning as commonly understood by those skilled in the art. It will be further understood that terms, such as those defined in commonly used dictionaries, should be interpreted as having a meaning that is consistent with their meaning in the context of the relevant art, and will not be interpreted in an idealized or overly formal sense unless expressly so defined herein.

The description made hereinabove is provided to describe the technical spirit of the present disclosure for the purpose of illustration only, and it is obvious to those skilled in the art that various modifications and changes may be made thereto without departing from the essential features of the present disclosure. Therefore, the embodiments disclosed herein are provided to describe the technical spirit of the present disclosure, but not intended to be limiting of the present disclosure, and the scope of technical spirit of the present disclosure is not limited by these embodiments. The scope of protection of the present disclosure should be interpreted by the appended claims, and the full technical spirit within its equivalent scope should be interpreted as falling in the scope of protection of the present disclosure.

1. A ring type module for taking a nasal tube inserted into a nasal cavity out of mouth to reposition a nasobiliary drainage tube from the mouth to nose, the ring type module comprising:
a ring part which is inserted into pharynx through an oral cavity; and
a ring handle connected to an end of the ring part,
wherein when an end of the nasal tube inserted into the pharynx through the nasal cavity passes through the ring part positioned in the pharynx, the nasal tube is taken out of the mouth by pulling the ring handle.

2. A device for oral-to-nasal repositioning of a nasobiliary drainage tube, comprising:
a ring type module which is inserted into an oral cavity to reposition the nasobiliary drainage tube from mouth to nose; and
a nasal tube which is inserted into pharynx through a nasal cavity, taken out of the mouth by the ring type module and connected with the nasobiliary drainage tube,
wherein the ring type module includes a snare shaped ring part which is inserted into the pharynx through the oral cavity, and a ring handle connected to an end of the ring part.

3. The device for oral-to-nasal repositioning of a nasobiliary drainage tube of claim 2, wherein the ring handle is provided in a shape of a flat bar.

4. The device for oral-to-nasal repositioning of a nasobiliary drainage tube of claim 2, wherein the ring part has a structure that is curved downward from an end of the ring handle.

5. The device for oral-to-nasal repositioning of a nasobiliary drainage tube of claim 2, further comprising:
an adjuster slidably moveably provided in the ring handle connected to the end of the ring part, to adjust a size of the ring part protruding from the end of the ring handle with sliding movement.

6. The device for oral-to-nasal repositioning of a nasobiliary drainage tube of claim 2, wherein the nasal tube is formed of a hollow elongated flexible tube, and has a multistep third mark at a point.

7. A method of using the device for oral-to-nasal repositioning of claim 2, the method comprising:
pushing and inserting the ring part of the ring type module into a user's mouth through a hole of a mouthpiece placed in the mouth;
pushing and inserting the nasal tube into nose to allow the flexible tube to pass through the ring part;
pulling the ring type module out of the mouth to take out the nasal tube caught in the ring part of the ring type module; and
connecting the nasobiliary drainage tube to the nasal tube pulled out as caught in the ring part and pulling the nasal tube out of the nose,
wherein in the insertion of the ring part into the mouth, a size of the ring part is adjusted using a length adjuster provided in the ring handle.

8. The device for oral-to-nasal repositioning of a nasobiliary drainage tube of claim 2, wherein the ring part formed approximately 8-12 cm in diameter.

9. The device for oral-to-nasal repositioning of a nasobiliary drainage tube of claim 4, the ring part is connected while being bent downward at the angle of approximately 15-30° from the end of the ring handle.

10. The device for oral-to-nasal repositioning of a nasobiliary drainage tube of claim 2, wherein the ring handle formed approximately 15 cm in length.
